(19) United States
(12) Patent Application Publication
(10) Pub. No.: US 2009/0125029 A1
(43) Pub. Date: May 14, 2009

(54) Device for Aligning and Guiding Femoral Resection Guide and Femoral Implant Impactor

(76) Inventors: Jai-Gon Seo, Seoul (KR); Eun-Suk Seo, Seoul (KR); Chong-Bum Kim, Seoul (KR)

Correspondence Address: PARK LAW FIRM, 3255 WILSHIRE BLVD, SUITE 1110, LOS ANGELES, CA, 90010, US

(21) Appl. No.: 12/264,481

(22) Filed: Nov. 4, 2008

(30) Foreign Application Priority Data
Nov. 8, 2007 (KR) 10-2007-113860

(51) Int. Cl. A61F 5/00; A61B 17/58

(52) U.S. Cl. 606/88; 606/87

(57) ABSTRACT

The present invention discloses a device for aligning and guiding a femoral resection guide and a femoral implant impactor, whereby the femoral resection guide and the femoral implant impactor are aligned perpendicular to the mechanical axes of the coronal plane and sagittal plane of the femur at the bottom end of the femur, and are guided so as to be rotation-aligned and mounted in parallel with a condyle axis connecting the inner condyle and outer condyle. The device comprises a main frame 110, joint bars 120 which are arranged extended downward from one end portion of the main frame 110 so as to be inserted and joined into the femoral resection guide 200 or femoral implant impactor 300, a support bar 130 which is arranged extended downward from the other end portion of the main frame 110, a first long bar 140 which is joined to the top surface of the one end portion of the main frame 110 to be arranged in parallel with the mechanical axis of the coronal plane of the femur, and a second long bar 150 which is joined to the support bar 130 to be arranged in parallel with the mechanical axis of the sagittal plane of the femur.

This application claims priority to Korean Patent Application No. 2007-113860, filed on Nov. 8, 2007, in the Korean Intellectual Property Office, the entire contents of which are hereby incorporated by reference.

### BACKGROUND OF THE INVENTION

1. Field of the Invention

The present invention relates to a device for aligning and guiding a femoral resection guide and a femoral implant impactor, and more specifically to a device for aligning and guiding a femoral resection guide and a femoral implant impactor, whereby the femoral resection guide and the femoral implant impactor are aligned perpendicular to the mechanical axes of the coronal plane and sagittal plane of the femur at the bottom end of the femur, and are guided so as to be rotation-aligned and mounted in parallel with a condyle axis connecting the inner condyle and outer condyle (hereinafter, refer as an inner and outer condyle axis).

2. Description of the Related Art

Knee arthroplasty has become one of the most commonly performed surgeries, in which a damaged or deformed knee joint resulting from congenital deformation, traumatic injuries, diseases, or degenerative arthritis, etc. is removed and replaced with an artificial joint. The most important thing in such an artificial knee joint replacement operation is to accurately resect the damaged knee joint and accurately mount the implant by considering the three-dimensional location and orientation of the normal knee joint in order to have smooth knee joint movement after an operation, prevent side effects such as arthrogryposis and causing pain, and prolong the life of the replaced artificial knee joint.

In the artificial knee joint replacement operation, the implant is mounted on the resected portion after resecting the knee joint. The following description will be centered on femoral resection related to the present invention and the mounting of femoral implant.

First, to describe femoral resection, as shown in FIG. 1, a femoral resection guide 10 has its top side arranged perpendicular to the mechanical axis of the femoral, and is rotated properly so as to be aligned in parallel with the inner and outer condyle axis, and then the resection guide is fixed to the femur. Here, the mechanical axis of a femur refers to an axis connecting the center of the femur head to the center of the bottom end of the femur, and since it cannot be seen the image is photographed by X-ray for the determination thereof. The rotation alignment of the condyle axis is preformed by rotating in parallel with the straight line connecting the vertexes of the inner and outer condyles confirmed by the naked eye or a palpation method during the surgical operation.

Subsequently, as shown in FIG. 2, a femur front bone resector is mounted on the femoral resection guide 10 to resect the femur front bone. Next, as shown in FIG. 3, a bottom end of the femur resector 30 is mounted on the femoral resection guide 10 and then the bottom end of the femur resector 30 is fixed to the femur by a pin P to remove the femoral resection guide 10. After that, as shown in FIG. 4, a cutter C is inserted into the resected bone of the bottom end of the femur resector 40 to resect the bottom end of the femur.

As can be seen in the above, the resection plane of the femur is decided according to the location and the rotation orientation in which the femoral resection guide 10 is mounted on the bottom end of the femur. Therefore, as is known in prior art, it is essential that the femoral resection guide 10 should be mounted perpendicular to the mechanical axis of the femur and in parallel with the inner and outer condyle axis.

Here, the mechanical axis of the femur can be described by division into a mechanical axis of coronal plane as a standing human body is seen frontally and a mechanical axis of sagittal plane as is seen laterally. Namely, as shown in FIG. 8 and FIG. 9, the mechanical axis of coronal plane refers to an axis projected in a line connecting the center of the femoral head A to the center B of the bottom end of the femur when the femur is projected in the Z direction, and the mechanical axis of sagittal plane refers to an axis projected in a line connecting the femur front-left part C and the outer condyle D with each other when the femur is projected in the X direction. And the inner and outer condyle axis refers to an axis projected in a line connecting the highest points of the inner and outer condyles with each other when the femur is projected in the Y direction, and this is also referred to as a rotation alignment line of the femoral transverse section.

Here, the femoral resection guide 10 should be mounted on the bottom end of femur perpendicular to the mechanical axis of the coronal plane and the mechanical axis of the sagittal plane respectively and aligned in parallel with the condyle axis. But there is no method in prior art for confirming whether the femoral resection guide 10 is aligned perpendicular to the mechanical axis of the coronal plane and the mechanical axis of the sagittal plane respectively and aligned in parallel with the inner and outer condyle axis and a method for correcting the error. Because of that, there is a problem that an ideal operation cannot be guaranteed.

Meanwhile, in a case of a femoral implant, as shown in FIG. 5, a femoral implant I with bone cement coated on the inner surface is mounted on the femoral impactor 30, and the femoral implant I is inserted into the bottom end of the femur. Next, when the femoral impactor 30 is hit, the bone cement coated on the inside of the femoral implant I comes into close contact with the bottom end of the femur to be hardened to complete an operation. At this time, the femoral implant I, based on the front surface thereof, should also be aligned perpendicular to the mechanical axis of the coronal plane and the mechanical axis of the sagittal plane of the femur respectively and in parallel with the inner and outer condyle axis. However, there is no method in prior art for confirming whether the femoral implant is aligned perpendicular to the mechanical axis of the coronal plane and the mechanical axis of the sagittal plane of the femur respectively and aligned in parallel with the inner and outer condyle axis and a method for correcting the error. Because of that, there is also a problem that an ideal operation cannot be guaranteed.

### SUMMARY OF THE INVENTION

Accordingly, it is an object of the present invention to provide a device for aligning and guiding a femoral resection guide and a femoral implant impactor whereby the femoral resection guide and femoral implant impactor are aligned perpendicular to the mechanical axes of the coronal and sagittal planes of the femur and are aligned in parallel with the inner and outer condyle axis of the transverse plane of the femur so as to be mounted on the bottom end of the femur.

In accordance with the present invention, there is provided a device for aligning and guiding a femoral resection guide and a femoral implant impactor comprising: a main frame; joint bars which are arranged extended downward from one end portion of the main frame so as to be inserted and joined into the femoral resection guide or femoral implant impactor; a support bar which is arranged extended downward from the other end portion of the main frame; a first long bar which is joined to the top surface of the one end portion of the main frame to be arranged in parallel with the mechanical axis of the coronal plane of the femur in order to confirm whether the femoral resection guide or the femoral implant impactor is aligned perpendicular to the mechanical axis of the coronal plane of the femur; and a second long bar which is joined to the support bar to be arranged in parallel with the mechanical axis of the sagittal plane of the femur in order to confirm whether the femoral resection guide or the femoral implant impactor is aligned perpendicular to the mechanical axis of the sagittal plane of the femur.

Preferably, the main frame, the first long bar and the second long bar are configured length-adjustably.

Preferably, the first holder with a through hole is mounted on the top surface of one end portion of the main frame, the second holder with a through hole is installed on one end portion of the support bar, and the support bar is installed slidably up or down in the second holder.

According to the present invention, the device for aligning and guiding a femoral resection guide and a femoral implant impactor guides the femoral resection guide and the femoral implant impactor in such a way that the femoral resection guide and the femoral implant impactor are mounted on the bottom end of the femur in a state aligned perpendicular to the mechanical axes of the coronal plane and the sagittal plane of the femur and aligned in parallel with the inner and outer condyle axis of the transverse plane of the femur, so that it has an effect that it can guide in such a way that the femoral resection guide and the femoral implant impactor are aligned three-dimensionally as a whole with respect to the femur.

### BRIEF DESCRIPTION OF THE DRAWINGS

These and other objects, features, and advantages of preferred embodiments of the present invention will be more fully described in the following detailed description, taken in conjunction with the accompanying drawings. In the drawings:

FIGS. 1 to 4 are schematic views for describing femoral resection processes;

FIG. 5 is a schematic perspective view for describing a mounting process of a femoral implant;

FIG. 6 is a perspective view of a device for aligning and guiding a femoral resection guide and a femoral implant impactor according to the present invention;

FIG. 7 is a partial sectional view of the device shown in FIG. 6; and

FIG. 8 and FIG. 9 are perspective views for describing the use of the device for aligning and guiding a femoral resection guide and a femoral implant impactor according to the present invention.

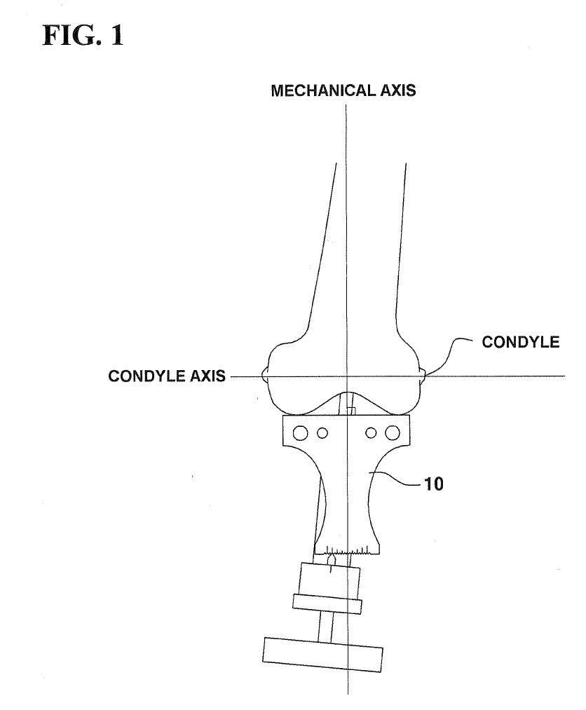

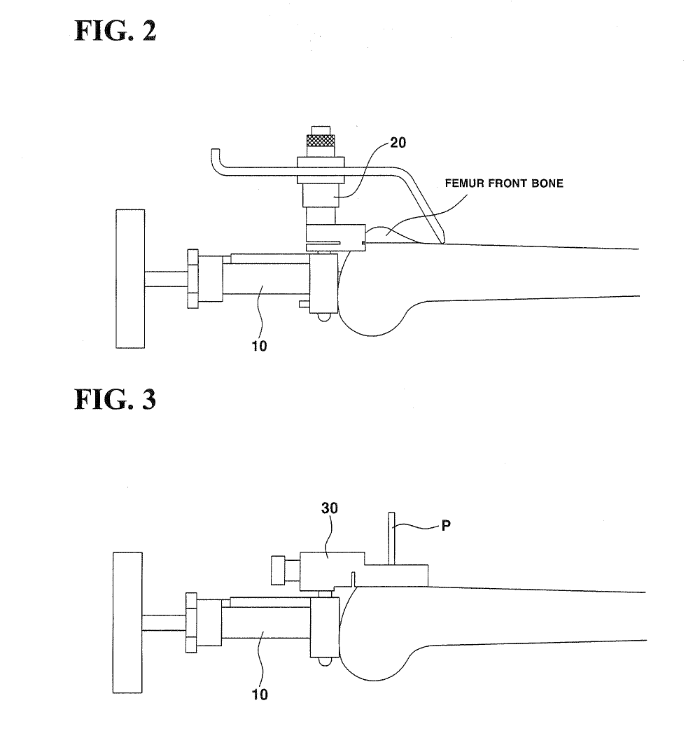

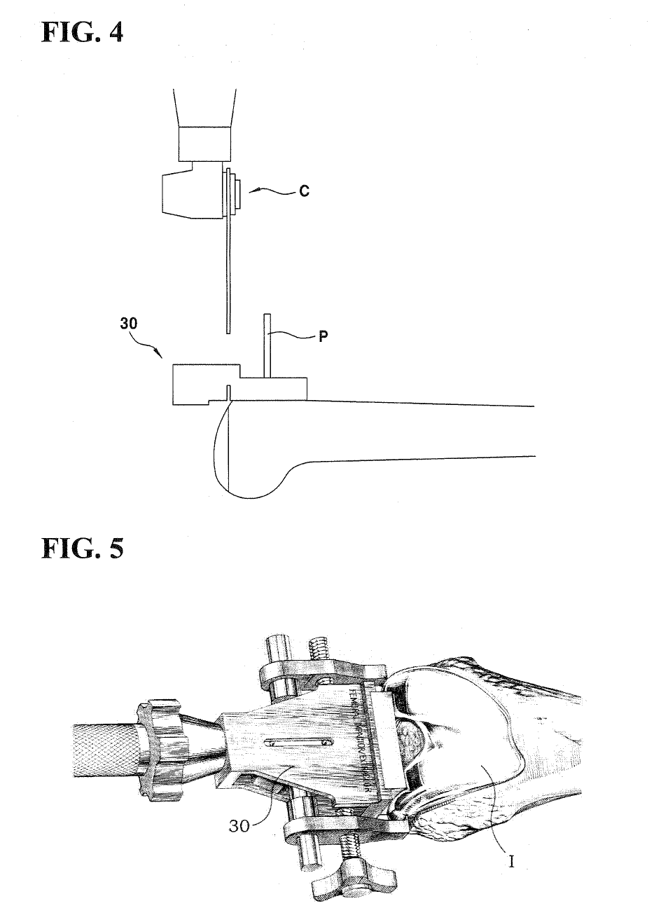

### DETAILED DESCRIPTION OF THE INVENTION

Below a device for aligning and guiding a femoral resection guide and a femoral implant impactor according to the present invention will be described in detail with reference to the accompanying drawings.

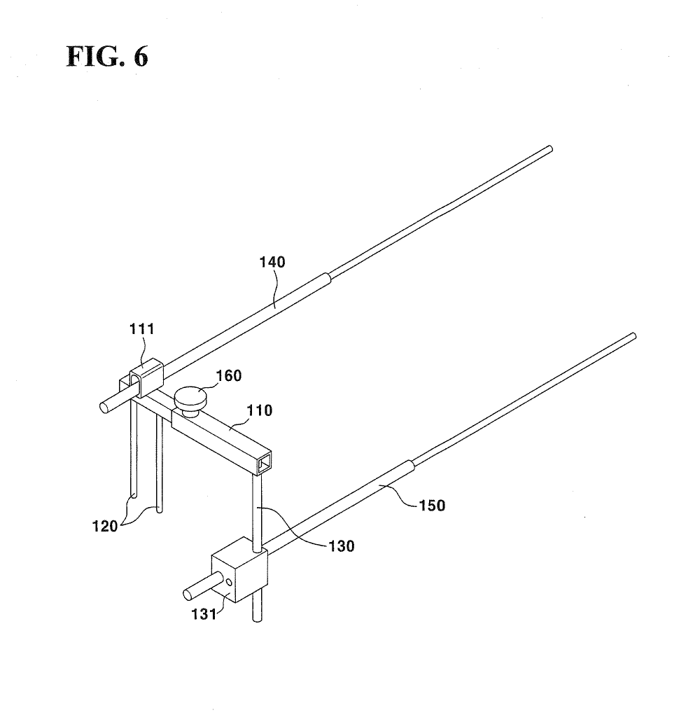

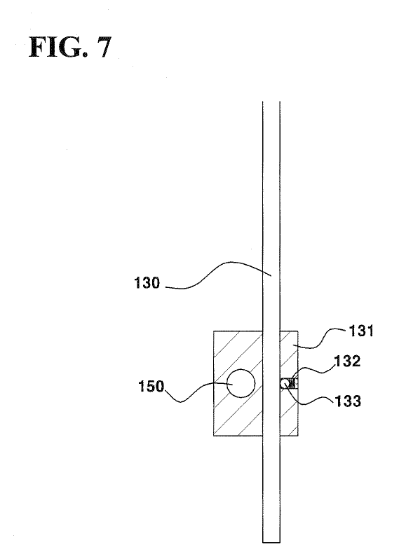

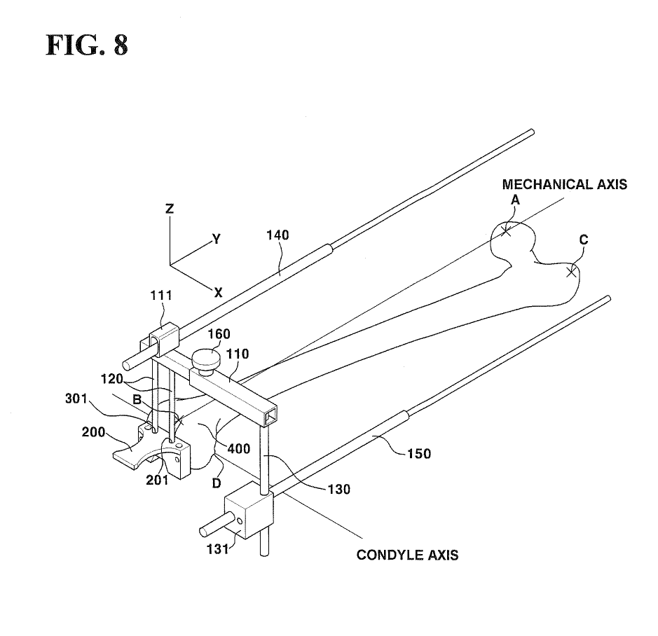

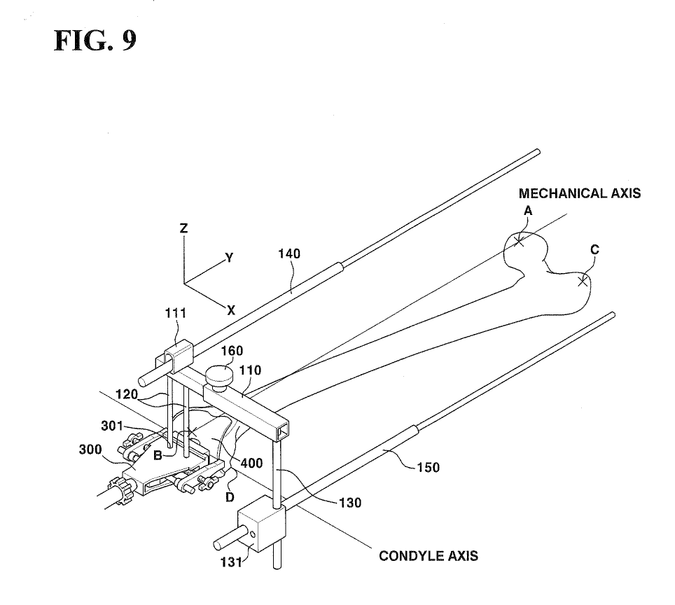

FIG. 6 is a perspective view of a device for aligning and guiding a femoral resection guide and a femoral implant impactor according to the present invention, FIG. 7 is a partial sectional view of the device shown in FIG. 6, and FIG. 8 and FIG. 9 are perspective views for describing the use of the device for aligning and guiding a femoral resection guide and a femoral implant impactor according to the present invention.

Referring to the drawings, the device for aligning and guiding a femoral resection guide and a femoral implant impactor according to the present invention comprises a main frame 110; joint bars 120 that are arranged extended downward from one end portion of the main frame 110 so as to be inserted and joined into the femoral resection guide 200 or femoral implant impactor 300; a support bar 130 that is arranged extended downward from the other end portion of the main frame 110; a first long bar 140 that is joined to the top surface of the one end portion of the main frame 110 to be arranged in parallel with the mechanical axis of the coronal plane of the femur in order to confirm whether the femoral resection guide 200 or the femoral implant impactor 300 is aligned perpendicular to the mechanical axis of the coronal plane of the femur; and a second long bar 150 that is joined to the support bar 130 to be arranged in parallel with the mechanical axis of the sagittal plane of the femur in order to confirm whether the femoral resection guide 200 or the femoral implant impactor 300 is aligned perpendicular to the mechanical axis of the sagittal plane of the femur.

The main frame 110 may be composed of one body, but it is preferable to be composed of two bodies, as shown in the views, so that the distance between the first long bar 140 and the second long bar 150 can be adjusted according to the size of the femur of the human body. Namely it is preferable to compose the main frame 110 in such a way that the frame of one side can be slidably inserted into the frame of the other side to make distance adjustment possible, and after the distance is adjusted, movement of the frames can be restrained by a clamp screw 160 as shown in the views.

The joint bars 120 are to be inserted and joined into the femoral resection guide 200 or the femoral implant impactor 300, and they are arranged extended downward from one end portion of the main frame 110. The joint bars 120 can be formed monolithically with the main frame 110 or can be composed by joining each other after they are made in separate members. In the femoral resection guide 200 or the femoral implant impactor 300 are punched joining holes 201 and 301, into which the bottom ends of the joint bars 120 are vertically inserted and joined thereto. But the present invention is not limited by such a joining method, and any person skilled in the art can understand that the joint bars can be joined to the femoral resection guide or femoral implant impactor in various methods other than this.

The support bar 130 is to adjust rotation alignment as well as support the second long bar 150, and is arranged extended downward from the other end portion of the main frame 110. The support bar 130 can also be formed monolithically with the main frame 110, or composed by joining each other after they are made in separate members.

As shown in FIGS. 8 and 9, the first long bar 140 is to confirm whether the femoral resection guide 200 or the impactor bar 300 for the femoral implant 400, based on the front surface thereof, are aligned perpendicular to the mechanical axis of the coronal plane of the femur. For this, it is joined to the top surface of one end portion of the main frame 110 to be arranged in parallel with the mechanical axis of the coronal plane of the femur. For the joining of the first long bar 140, a first holder 111 with a through hole formed is mounted on the top surface of one end portion of the main frame 110. And a marking bar (not shown) is attached to the outside of the skin of the vertical upper portion corresponding to the mechanical axis of the coronal plane of the femur in the center of the femoral head A. Accordingly, if the first long bar 140 is inserted into the through hole of the first holder 111 in the direction of the femoral head A and then fixed to the marking bar, it is possible to confirm whether the first long bar 140 is aligned in parallel to the mechanical axis of the coronal plane of the femur.

As shown in FIGS. 8 and 9, the second long bar 150 is to confirm whether the femoral resection guide 200 or the impactor 300 for the femoral implant 400, based on the front surface thereof, are aligned perpendicular to the mechanical axis of the sagittal plane of the femur. For this, it is joined to the support bar 130 and is arranged in parallel with the mechanical axis of the sagittal plane of the femur. For the joining of the second long bar 150, a second holder 131 with a through hole is installed on one end portion of the support bar 130. The second holder 131 is installed in a location corresponding to the outer condyle D of the femur. And a marking bar (not shown) is attached on the side of the skin corresponding to the mechanical axis of the sagittal plane of the femur in the femur front-left part C. Accordingly, if the second long bar 150 is inserted into the through hole of the second holder 131 in the direction of the femur front-left part C and then fixed to the marking bar, it is possible to confirm whether the second long bar 150 is aligned to the mechanical axis of the sagittal plane of the femur.

Meanwhile, it is preferable that the support bar 130 is installed slidably up or down in the second holder 131. As an example of such a configuration, a mounting hole is formed in the second holder 131, and in this mounting hole is mounted a ball 133 that is pressed by a spring 132. The ball 133 presses the support bar 130 by the elastic force of the spring 132. Accordingly, at normal times the support bar 130 is pressed by the ball 133 pressed by the spring 132 so as to maintain the stopped state, but if the support bar 130 is pushed up or down, it overcomes the pressing force of the spring 132 to push the ball 133 inward of the mounting hole, so that up and down sliding movement of the support bar 130 becomes possible.

The device for aligning and guiding a femoral resection guide and a femoral implant impactor configured like this is, as will be described later, used in aligning the first long bar 140 to the mechanical axis of the coronal plane and then aligning the second long bar 150 to the mechanical axis of the sagittal plane.

First, as shown in FIGS. 8 and 9, the joint bars 120 are joined to the femoral resection guide 200 or femoral implant impactor 300. Then, the first long bar 140 is inserted into the first holder 111, and pushed out toward the femoral head A. Next, the first long bar 140 is arranged in agreement with the mechanical axis of the coronal plane that connects the center of the femoral head A and the femoral bottom end center B. At this time, the outward end portion of the first long bar 140 is fixed to the marking bar (not shown) attached to the outside of the skin of the vertical upper portion corresponding to the mechanical axis of the coronal plane of the femur in the center of the femoral head A. Then, the femoral resection guide 200 or the femoral implant impactor 300 also can be aligned perpendicular to the mechanical axis of the coronal plane by the first long bar 140 that is arranged in agreement with the mechanical axis of the coronal plane of the femur.

Meanwhile, in the state as described above, the first long bar 140 is aligned with the mechanical axis of the coronal plane of the femur, so that the rotation alignment of the condyle axis defined by an angle of 90 degrees with the mechanical axis of the coronal plane of the femur is made naturally.

Next, the second long bar 150 is inserted into the second holder 131 to be pushed out toward the femur front-left part C. Next, the outward end portion of the second long bar 150 is fixed to the marking bar (not shown) attached to the side of the skin corresponding to the mechanical axis of the sagittal plane in the femur front-left part C. Then, the second long bar 150 becomes the state in which the inward end portion is inserted into the second holder 131, and the outward end portion becomes the state of being fixed by the marking bar attached to the side of the skin corresponding to the mechanical axis of the sagittal plane of the femur in the femur front-left part C. In this state, the second holder 131 is moved by sliding up and down on the support bar 130 to make the second long bar 150 in agreement with the mechanical axis of the sagittal plane of the femur, and then the second holder 131 is fixed to the support bar 130.

In such a state, the femoral resection guide 200 or the femoral implant impactor 300 is aligned to the mechanical axis of the coronal plane and the mechanical axis of the sagittal plane of the femur by the first long bar 140 aligned to the mechanical axis of the coronal plane and the second long bar 150 aligned to the mechanical axis of the sagittal plane of the femur. The first long bar 140 is aligned to the mechanical axis of the coronal plane, so that the rotation alignment of the condyle axis defined by an angle of 90 degrees with the mechanical axis of the coronal plane of the femur is also made naturally. Accordingly, the femoral resection guide 200 or the femoral implant impactor 300 as a whole can be mounted on the bottom end of the femur in a state in which the mechanical axis alignment of the coronal plane of the femur, the mechanical axis alignment of the sagittal plane of the femur and the rotation alignment of the condyle axis, namely the three-axis alignment, are accomplished.

Although the present invention has been described in detail reference to its presently preferred embodiment, it will be understood by those skilled in the art that various modifications and equivalents can be made without departing from the spirit and scope of the present invention, as set forth in the appended claims.

What is claimed is:

1. A device for aligning and guiding a femoral resection guide and a femoral implant impactor comprising:
a main frame;
joint bars which are arranged extended downward from one end portion of the main frame so as to be inserted and joined into the femoral resection guide or femoral implant impactor;
a support bar which is arranged extended downward from the other end portion of the main frame;
a first long bar which is joined to the top surface of the one end portion of the main frame to be arranged in parallel with the mechanical axis of the coronal plane of the femur in order to confirm whether the femoral resection guide or the femoral implant impactor is aligned perpendicular to the mechanical axis of the coronal plane of the femur; and
a second long bar which is joined to the support bar to be arranged in parallel with the mechanical axis of the sagittal plane of the femur in order to confirm whether the femoral resection guide or the femoral implant impactor is aligned perpendicular to the mechanical axis of the sagittal plane of the femur.

2. The device according to claim 1, wherein the main frame, the first long bar and the second long bar are configured length-adjustably.

3. The device according to claim 1, wherein the first holder with a through hole is mounted on the top surface of one end portion of the main frame, the second holder with a through hole is installed on one end portion of the support bar, and the support bar is installed slidably up or down in the second holder.
